(19) United States
(12) Patent Application Publication
(10) Pub. No.: US 2022/0009958 A1
(43) Pub. Date: Jan. 13, 2022

(54) METHODS OF PURIFICATION OF ALBUMIN FUSION PROTEINS

(71) Applicant: Novo Nordisk A/S, Bagsvaerd (DK)

(72) Inventors: Viveka Dolby, Holte (DK); Are Bogsnes, Nivaa (DK)

(21) Appl. No.: 17/487,359

(22) Filed: Sep. 28, 2021

Related U.S. Application Data
(63) Continuation of application No. 16/605,591, filed on Oct. 16, 2019.

(30) Foreign Application Priority Data
Apr. 20, 2017 (EP) 17167261.1

(51) Int. Cl. C07K 1/18; B01D 15/16; B01D 15/36; B01D 15/42; C07K 14/475; C07K 14/765

(52) U.S. Cl. CPC C07K 1/18; B01D 15/166; B01D 15/362; C07K 2319/31; C07K 14/475; C07K 14/765; B01D 15/426

(57) ABSTRACT

The present invention provides a chromatographic separation method for improving the quality of albumin fusion protein solutions by removing impurities from the albumin fusion protein solution. This invention provides albumin fusion protein solution with a significantly reduced amount of the (yellow) coloured impurities and HCP.

### CROSS-REFERENCE TO RELATED APPLICATIONS

This application is continuation of U.S. application Ser. No. 16/605,591, filed Oct. 16, 2019, which is a § 371 National Stage application of International Application PCT/EP2018/060033 (published as WO 2018/193033), filed Apr. 19, 2018, which claims priority to European Patent Application 17167261.1, filed Apr. 20, 2017; the contents of which are incorporated herein by reference.

### TECHNICAL FIELD OF INVENTION

The present invention relates to the field of protein purification and particularly, it relates to methods of removing impurities from albumin fusion protein solutions.

### BACKGROUND OF INVENTION

Albumin fusion proteins have been well-studied and widely applied in biopharmaceutics. A common application of albumin fusion proteins is to extend the plasma half-life of therapeutic proteins and peptides. An example of this is Macrophage Inhibitory Cytokine-1 (MIC-1) albumin fusion proteins, for example described in WO/2015/197446 and WO 2015/198199.

Albumin fusion protein solutions often contain impurities that can affect quality and visual appearance of the albumin fusion protein product. Coloured impurities, often yellow coloured impurities, are in particular associated with albumin fusion protein solutions.

Methods such as heat treatment, hydrophobic interaction chromatography (HIC), active coal filtering (US2011027221), etc. have previously been employed in the attempt to improve quality and visual appearance of albumin solutions. These methods are, however, not sufficiently efficient and there is a need for alternative purification methods.

### SUMMARY OF INVENTION

The present invention provides a method for improving the quality of albumin fusion protein solutions, by reducing the content of impurities. In one aspect, the present invention relates to a chromatographic separation method for removing impurities, such as yellow coloured impurities and host cell proteins (HCP) impurities from albumin fusion protein solutions.

In one aspect, the method of purification of aqueous albumin fusion protein solution, of the invention, comprises the following steps: (i) loading said fusion protein solution onto a column comprising a cationic exchange chromatographic resin, and (ii) eluting the fusion protein from the column with a cation containing gradient.

In one embodiment, the albumin fusion protein is a MIC-1 human serum albumin fusion protein (HSA-MIC-1).

In one embodiment, the cation containing gradient is a Ca²⁺ containing gradient.

In one embodiment, the elution buffer has a pH of 4.0-5.0. In further embodiments the elution buffer has a pH of 4.6, 4.5, 4.4, 4.3, 4.2, or 4.0.

In one embodiment, the impurities removed comprise yellow coloured impurities.

In one aspect, the present invention relates to a method of production of an albumin fusion protein wherein the method comprises the steps of:

(i) expressing an albumin fusion protein in a solution, and

(ii) loading said fusion protein solution onto a column comprising a cationic exchange chromatographic resin, and (ii) eluting the fusion protein from the column with a gradient of increasing Ca²⁺ concentration.

In one embodiment, the albumin fusion protein of the method of production is a MIC-1 human serum albumin fusion protein.

The industrial implications of the present invention relate to the optimisation of protein purification. It is the purpose of the optimised purification to achieve an end product to be used commercially comprising albumin fusion protein solutions with a reduced and better controlled content of impurities, particularly yellow coloured impurities and HCP impurities.

### BRIEF DESCRIPTION OF DRAWINGS

FIG. 1 shows elution at different pH values. Retention time increased at lower pH improving reduction of HCP (as described in Example 6).

### DETAILED DESCRIPTION OF INVENTION

The inventors of the present invention have developed a method for efficient removal of impurities, such as HCP and yellow coloured impurities, from albumin fusion protein solutions.

In one aspect, the present invention concerns a purification step in which albumin fusion protein solution is loaded onto a cationic exchange chromatographic resin, and a significant amount of HCP and yellow coloured impurities are separated and removed during elution with a cation containing gradient.

### Albumin Fusion Protein

Albumin fusion proteins herein are proteins created through the in-frame joining of two or more DNA sequences which originally encoded an albumin protein and at least one different type of protein or peptide, optionally separated by a linker. Translation of the fusion protein DNA sequence will result in a single protein sequence which may have functional properties derived from each of the original proteins or peptides. The resulting fusion protein DNA sequence may be inserted into an appropriate expression vector that supports the heterologous fusion protein expression in a standard host organism.

Human Serum Albumin (HSA) is a plasma protein of about 66 500 Da and is comprised of 585 amino acids, including at least 17 disulfide bridges. (Peters, T, jr. (1996), All about Albumin: Biochemistry, Genetics and Medical, Applications, p 10, Academic Press, inc., Orlando (ISBN 0-12-552110-3). HSA has inherent properties such as high solubility and stability which makes it beneficial to use as fusion partner for improving expression yield and conferring stability to various therapeutic proteins. Human serum albumin as fusion partner herein may also increase the plasma half-life of therapeutic proteins by significant size increase, which inhibits renal clearance and/or by binding the Fc Neonatal Receptor, which allows recycling from the endosome and prevention of lysomal degradation allowing the therapeutic protein to be present longer in circulation.

Albumin, preferably human serum albumin herein may thus be fused with various therapeutic proteins such as e.g. a coagulation factor (e.g. Factor VII, Factor VIII, or Factor IX), a human growth hormone, an insulin, a GLP-1, peptides such as e.g. a macrophage inhibitory cytokine-1 (MIC-1), etc. For example, PCT publications WO01/79271 and WO03/59934 disclose albumin fusion proteins comprising a variety of therapeutic proteins.

In an aspect of the invention, the albumin fusion protein is albumin fused to MIC-1 (HSA-MIC-1). For example, PCT publications WO2015/197446 and WO2015/198199 disclose MIC-1 albumin fusion proteins and methods for their production. In an embodiment of the invention, the albumin fusion protein comprises a human serum albumin or a functional variant thereof and a human MIC-1 or a functional variant thereof (HSA-MIC-1).

The term “MIC-1” as used herein means Macrophage Inhibitory Cytokine-1 (MIC-1), also known as Growth Differentiation Factor 15 (GDF-15), placental bone morphogenetic protein (PLAB) and nonsteroidal anti-inflammatory drug-activated gene (NAG-1). MIC-1 was first described in 1997 (Bootcov et al, Proc. Natl. Acad. Sci. October 1997) based on experiments showing increased expression in activated macrophages. MIC-1 is synthesized as a 62 kDa intracellular homodimer precursor protein which subsequently is cleaved by a furin-like protease into a 24.5 kDa homodimer. The sequence of the full length wild type human MIC-1 is available from the UNIPROT database with accession no. Q99988.

Albumin fusion proteins of the present invention may be produced by means of recombinant protein technology known to persons skilled in the art. In general, nucleic acid sequences encoding the fusion protein of interest are modified to encode the desired fusion protein. This modified sequence is then inserted into an expression vector, which is in turn transformed or transfected into the expression host cells. The desired fusion protein is then expressed in the host cells and subsequently recovered and purified.

Proteins solutions herein are preferably aqueous albumin fusion protein solutions comprising at least 90% water and preferably no or only small amounts of organic solvents (less than 1%).

### Ion Exchange Chromatography

Ion-exchange chromatography is a chromatography process that separates ions and polar molecules in solution based on their affinity to an ion exchanger. Water-soluble and charged molecules bind to oppositely charged moieties by forming non-covalent bonds to the insoluble stationary phase. An equilibrated stationary phase in a column comprises an ionizable functional group where the targeted molecules to be separated can bind while passing the solution through the column.

The starting material for the purification step of the present invention may be any aqueous solution comprising albumin fusion proteins. The starting material may have been subjected to one or more purification or chemical modification steps prior to the purification according to the invention. In one embodiment the starting material has been subjected to purification by anion exchange chromatography prior to the cation exchange chromatography method according to the invention.

Following loading of the albumin fusion protein solution on the anion exchange column, the albumin protein can be eluted in a Tris buffered sodium chloride solution at pH 7.7, following a wash containing ethanol and Ca²⁺.

Cation exchange process: The cation exchange resin packed in a column is equilibrated with an equilibration buffer. The albumin fusion protein solution is loaded onto the column, diluted to a conductivity and pH similar to equilibration buffer. The albumin fusion protein is then eluted from column in a gradient from the equilibration buffer to the elution buffer.

Buffer systems: A suitable type of buffer system herein includes a combination of e.g. acetic acid and NaOH selected to maintain a low pH (pH value of about 4.0-5.0) in the equilibrium buffer/solution and/or the elution buffer. This buffer system can be used to adjust pH of e.g. elution buffers and washing/equilibrium buffers herein.

Equilibration/Wash buffer: One example of a suitable Equilibration/Wash buffer herein is: 20 mM Acetic acid, 10 mM NaOH, 100 mM NaCl, pH 4.6

Elution buffer: Albumin fusion protein solutions can be eluted using a cation gradient. Types of suitable cations include Na⁺, Mg²⁺ and Ca²⁺, NH₄⁺, K⁺.

Preferred cation counter ions include chloride and acetate.

Preferred ion combination is CaCl₂).

One example of a suitable eluent herein is: 20 mM Acetic acid, 16 mM NaOH, 500 mM CaCl₂), pH 4.7.

This eluate is suitable for further purification on e.g. hydrophobic interaction chromatography (HIC).

### Removing Impurities

The method of the present invention is efficient in reducing the content of impurities, such as yellow coloured impurities and host cell proteins (HCP), from albumin fusion protein solutions.

The term “yellow coloured impurities” as used herein includes, within the meaning thereof, not only culture medium-derived colouring contaminants but also any and all substances capable of yellow colouring fusion protein solutions. Yellow or dark yellowish brown coloured impurities are in particular associated with albumin fusion protein solutions. These impurities cannot be removed to a satisfactory extent by known methods of purifying albumin fusion protein solutions.

When performing the purification method of the invention, a part of the yellow colour impurities does not bind to the column and appears in the flow through during load. Another part of the yellow colour impurities elutes later in the gradient, thus separated from the albumin fusion protein. Furthermore, another part is bound tightly to the column and is removed during alkaline regeneration of the column.

The term “removing” or “reducing” as used herein in context of the method of the present invention means removing a certain content/amount of impurities/contaminants, particularly yellow coloured and HCP impurities, from the fusion protein solution; i.e. of reducing the content/amount of impurities in the fusion protein solution being subject to the method of the present invention.

The level of yellow coloured impurities can be measured by means of absorption at certain wavelengths. The purified sample was subjected to absorbance measurements at the wavelengths of 280 nm (protein) and 470 nm (yellow colour), and the absorbance ratio between 470 nm and 280 nm multiplied with 1000 was calculated as an indication of colour relative to protein. A reduction factor was calculated by dividing the value prior to purification (load) by the value after purification (eluate).

The term “host cell proteins (HCP)” as used herein in context of the method of the present invention is defined as proteins produced or encoded by the host organism (in this case CHO cells) and unrelated to the intended recombinant protein.

HCP impurities can be measured by ELISA and the fold reduction over the purification can be calculated by dividing the value prior to purification (load) by the value after purification (eluate).

### EXAMPLES

In the examples below purification of MIC-1 fused to human serum albumin (HSA-MIC-1) expressed in mammalian cells (CHO cells) are exemplified.

### Example 1

Pre-treatment of load: The protein solution was partly purified on an anion exchange resin eluted in a Tris buffered sodium chloride solution at pH ˜7.7, following a wash containing ethanol and Ca²⁺.

Prior to load on cation exchange column, the HSA-MIC1 solution was subjected to virus inactivation by adding octanoate to a final concentration of about 10 mM and adjusting the pH to about 4.9. The protein solution was left for inactivation for approximately 30 minutes and then diluted with water to a conductivity of approximately 11 mS/cm, pH adjusted to 4.6.

A 99 ml column packed with POROS 50HS was equilibrated with 297 ml 20 mM Acetic acid, 10 mM NaOH, 100 mM NaCl, pH 4.6. The column was loaded with 375 ml partly purified protein solution, containing approximately 1.6 g HSA-MIC-1, and then followed by a wash with 297 ml equilibration buffer. The column was eluted in a gradient over 12 column volumes. Start condition was 90% equilibration buffer+10% elution buffer (20 mM Acetic acid, 16 mM NaOH, 500 mM CaCl₂, pH 4.7) and final condition was 10% equilibration buffer and 90% elution buffer.

TABLE 1

| | | | A470 nm/ | Colour | HCP |
|---|---|---|---|---|---|
| | | | A280 nm | reduction | reduction |
| Fraction | A280 nm | A470 nm | *1000 | factor | factor |
| Load | 1.983 | 0.099 | 49.9 | — | — |
| Eluate | 0.334 | 0.004 | 12.0 | 4.2 | 3.1 |

The method carried out as described above gave a reduction of 4.2 fold with regard to yellow colour and 3.1 fold with regard to host cell proteins (HCP), as shown in Table 1.

### Example 2

Pre-treatment of load: The protein solution was partly purified on an anion exchange resin eluted in a Tris buffered sodium chloride solution at pH ˜7.7, following a wash containing ethanol and Ca²⁺.

Prior to load on cation exchange column, the HSA-MIC1 solution was subjected to virus inactivation by adding octanoate to a final concentration of about 10 mM and adjusting the pH to about 4.9. The protein solution was left for inactivation for approximately 30 minutes and then diluted with water to a conductivity of approximately 15 mS/cm, pH adjusted to 4.2.

A 39.25 ml column packed with POROS 50HS was equilibrated with 117.75 ml 20 mM acetic acid, 5.8 mM NaOH, 150 mM NaCl, pH 4.2. The column was loaded with 109.5 ml partly purified protein solution, containing approximately 0.6 g mg HSA-MIC-1, and then followed by a wash with 117.75 ml equilibration buffer. The column was eluted in a gradient over 10 column volumes. Start condition was 90% equilibration buffer+10% elution buffer (20 mM acetic acid, 11 mM NaOH, 500 mM CaCl₂, pH 4.2) and final condition was 10% equilibration buffer and 90% elution buffer.

TABLE 2

| | | | A470 nm/ | Colour | HCP |
|---|---|---|---|---|---|
| | | | A280 nm | reduction | reduction |
| Fraction | A280 nm | A470 nm | *1000 | factor | factor |
| Load | 2.008 | 0.099 | 49.3 | — | — |
| Eluate | 0.300 | 0.002 | 6.7 | 7.4 | 5.2 |

The method carried out as described above gave a reduction of 7.4 fold with regard to yellow colour and 5.2 fold with regard to host cell proteins (HVP), as shown in Table 2.

### Example 3

Pre-treatment of load: The protein solution was partly purified on an anion exchange resin eluted in a Tris buffered sodium chloride solution at pH ˜7.7, following a wash containing ethanol and Ca²⁺.

Prior to load on cation exchange column, the HSA-MIC1 solution was subjected to virus inactivation by adding octanoate to a final concentration of about 10 mM and adjusting the pH to about 4.9. The protein solution was left for inactivation for approximately 30 minutes and then diluted with water to a conductivity of approximately 11 mS/cm, pH adjusted to 4.5.

A 8.6 ml column packed with POROS 50HS was equilibrated with 25.8 ml 20 mM acetic acid, 10 mM NaOH, 100 mM NaCl, pH 4.6. The column was loaded with 28.6 ml partly purified protein solution, containing approximately 156 mg HSA-MIC-1, and then followed by a wash with 28.6 ml equilibration buffer. The column was eluted in a gradient over 12 column volumes. Start condition was 90% equilibration buffer+10% elution buffer (20 mM acetic acid, 18 mM NaOH, 1000 mM NaCl, pH 4.8) and final condition was 10% equilibration buffer and 90% elution buffer.

### Results:

The method carried out as described above gave a similar reduction of yellow colour upon visual evaluation.

### Example 4

Pre-treatment of load: The protein solution was partly purified on an anion exchange resin eluted in a Tris buffered sodium chloride solution at pH ˜7.7, following a wash containing ethanol and Ca²⁺.

Prior to load on cation exchange column, the HSA-MIC1 solution was subjected to virus inactivation by adding octanoate to a final concentration of about 10 mM and adjusting the pH to about 4.9. The protein solution was left for inactivation for approximately 30 minutes and then diluted with water to a conductivity of approximately 11 mS/cm, pH adjusted to 4.5.

A 5.7 ml column packed with POROS 50HS was equilibrated with 17.1 ml 20 mM acetic acid, 10 mM NaOH, 100 mM NaCl, pH 4.6. The column was loaded with 19.4 ml partly purified protein solution, containing approximately 90 mg HSA-MIC-1, and then followed by a wash with 17.1 ml equilibration buffer. The column was eluted in a gradient over 12 column volumes. Start condition was 90% equilibration buffer+10% elution buffer (20 mM acetic acid, 16 mM NaOH, 500 mM MgCl₂, pH 4.9) and final condition was 10% equilibration buffer and 90% elution buffer.

TABLE 3

| | | | A470 nm/ | Colour | HCP |
|---|---|---|---|---|---|
| | | | A280 nm | reduction | reduction |
| Fraction | A280 nm | A470 nm | *1000 | factor | factor |
| Load | 1.942 | 0.129 | 66.4 | — | — |
| Eluate | 0.306 | 0.003 | 9.8 | 6.8 | 4.6 |

The method carried out as described above gave a reduction of 6.8 fold with regard to yellow colour and 4.6 fold with regard to host cell proteins (HCP), as shown in Table 3.

### Example 5

42 ml of a partly purified clearly yellow sample from an anion exchange column (GigaCap Q 650M) was concentrated approximately 5 times, to 9 ml, on an Amicon Ultra filter with a molecular weight cut-off of 30K. The sample was dissolved in a buffer containing 500 mM NaCl, 20 mM Tris, 15 mM HCl (pH 7.7). The permeate had no colour, whereas the retentate gained increasingly more colour upon concentration. The experiment was repeated after adjustment with 11 mM acetic acid, to pH 4.5. Also at this condition, no removal of yellow colour was observed.

### Example 6

Pre-treatment of load: The protein solution was partly purified on an anion exchange resin eluted in a Tris buffered sodium chloride solution at pH ˜7.7, following a wash containing ethanol and Ca²⁺.

Prior to load on cation exchange column, pH in the load samples was diluted with water and adjusted with acetic acid to 4.6, 4.5, 4.4, 4.3, 4.2, and 4.0, respectively.

A 5.7 ml column packed with POROS 50HS was equilibrated with 17.1 ml buffer consisting of 100 mM NaCl, 20 mM acetic acid, adjusted with NaOH to the varying pH values from pH 4.6 to pH 4.0. The column was loaded with the sample, and then followed by a wash with 17.1 ml equilibration buffer. The column was eluted in a gradient over 12 column volumes. Start condition was 90% equilibration buffer+10% elution buffer (500 mM CaCl₂, 20 mM acetic acid, adjusted with NaOH to the varying pH values from pH 4.6 to 4.0) and final condition was 10% equilibration buffer and 90% elution buffer.

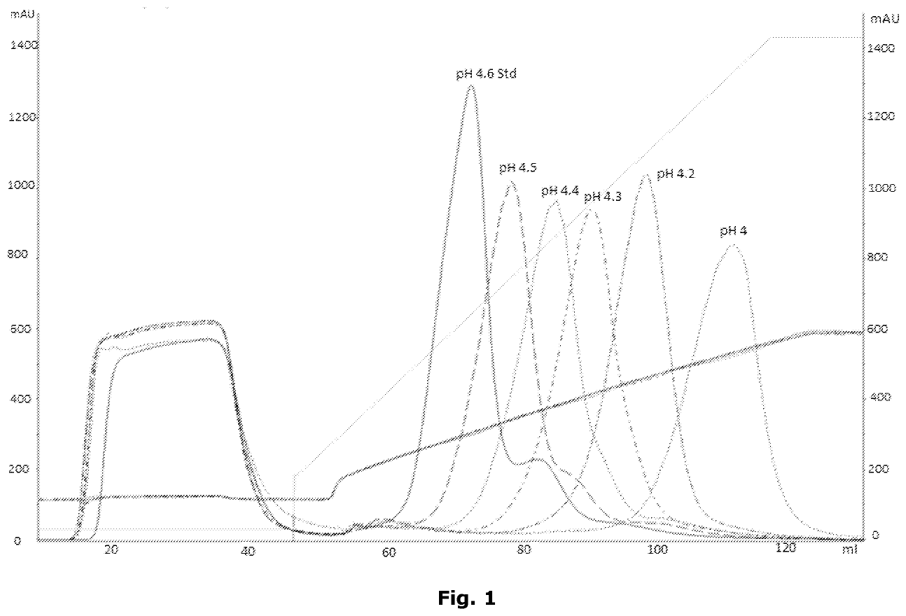

Lower pH resulted in longer retention time and generally an increasing HCP reduction factor, as shown in Table 4 and FIG. 1.

TABLE 4

| pH | HCP reduction factor |
|---|---|
| 4.6 | 3.8 |
| 4.5 | 3.1 |
| 4.4 | 3.7 |
| 4.3 | 4.0 |
| 4.2 | 7.8 |
| 4.0 | 6.5 |

1. A method of purification of an aqueous albumin fusion protein solution, wherein said method comprises: (i) the step of loading said fusion protein solution onto a column comprising a cationic exchange chromatographic resin, and (ii) the step of eluting the fusion protein from the column using an elution buffer with a cation containing gradient.

2. The method according to claim 1, wherein the albumin fusion protein is a MIC-1 human serum albumin fusion protein.

3. The method according to claim 1, wherein the cation containing gradient is a Na⁺, Mg²⁺ NH₄⁺, K⁺ or Ca²⁺ containing gradient.

4. The method according to claim 3, wherein the cation containing gradient is a Ca²⁺ containing gradient.

5. The method according to claim 1, wherein the elution buffer has a pH of 4.0-5.0.

6. The method according to claim 5, wherein the elution buffer has a pH of 4.2.

7. The method according to claim 1, wherein the albumin fusion protein is eluted from the cation exchange column using an elution buffer gradient comprising 20 mM acetic acid, 11 mM NaOH and 500 mM CaCl₂), at a pH of 4.2 and gradually increasing the content of CaCl₂) in the elution buffer to 500 mM at pH 4.2.

8. The method according to claim 1, further comprising carrying out an anion exchange purification step of the albumin fusion protein solution prior to step (i).

9. The method according to claim 8, wherein the albumin fusion protein is eluted from an anion exchange column in a Tris buffered sodium chloride solution at pH of 7.7, following a wash comprising ethanol and Ca²⁺.

10. The method according to claim 1, further comprising, after step (ii), a step of washing the column with a buffer comprising 20 mM acetic acid, 10 mM NaOH, and 100 mM NaCl, at a pH of 4.6.

11. The method according to claim 1, wherein the content of yellow coloured impurities is reduced at least 3.0 fold as measured by absorbance ratio between 470 nm and 280 nm.

12. The method according to claim 1, wherein the content of HCP impurities is reduced at least 3.0 fold as measured by ELISA.

13. A method of production of an albumin fusion protein comprising the method of purification of an albumin fusion protein solution according to claim 1.
